(19) United States
(12) Patent Application Publication
(10) Pub. No.: US 2023/0380684 A1
(43) Pub. Date: Nov. 30, 2023

(54) SYSTEMS AND METHODS FOR IMPROVED EYE-TRACKING DEVICES AND ASSOCIATED USER INTERFACES

(71) Applicant: EyeTech Digital Systems, Inc., Tempe, AZ (US)

(72) Inventors: Robert Chappell, Mesa, AZ (US); Juan Zoppetti, Mesa, AZ (US); Araz Vartanian, Scottsdale, AZ (US); Caleb Hinton, Mesa, AZ (US); Jessica Bruny, Tempe, AZ (US); Kiuanta Canteen, Tempe, AZ (US); Kevin Forde-Nihipali, Tempe, AZ (US); Jessica Williams, Tempe, AZ (US)

(21) Appl. No.: 18/325,841

(22) Filed: May 30, 2023

Related U.S. Application Data
(60) Provisional application No. 63/346,433, filed on May 27, 2022.
(60) Provisional application No. 63/358,601, filed on Jul. 6, 2022.
(60) Provisional application No. 63/358,603, filed on Jul. 6, 2022.
(60) Provisional application No. 63/358,606, filed on Jul. 6, 2022.
(60) Provisional application No. 63/388,376, filed on Jul. 12, 2022.
(60) Provisional application No. 63/395,578, filed on Aug. 5, 2022.
(60) Provisional application No. 63/395,580, filed on Aug. 5, 2022.
(60) Provisional application No. 63/396,448, filed on Aug. 9, 2022.
(60) Provisional application No. 63/397,075, filed on Aug. 11, 2022.

(51) Int. Cl. A61B 3/113; G16H 40/67; G16H 80/00; G06V 40/18; G06V 10/141; G06V 10/82

(52) U.S. Cl. CPC A61B 3/113; G16H 40/67; G16H 80/00; G06V 40/193; G06V 10/141; G06V 10/82

(57) ABSTRACT

An eye-tracking device and associated software together allow a patient to communicate with health-care providers (HCPs), family members, and other individuals via eye gaze technology. The system presents the patient with a hierarchical and intuitive set of graphical menus that, for example, allow the patient, via his or her eye-gaze location, to indicate basic needs (e.g., food, bathroom, bed adjustment), alert the nursing staff to an emergency situation, and/or indicate pain level associated with a specific part of the patient's body. In parallel, an administrator of the system may use traditional touch screen functionality to assist in calibration, select patient preferences, and otherwise configure the system for use. In yet another embodiment, improved systems and methods are provided for performing eye-tracking using a variety of machine learning techniques instead of, or in addition to, traditional geometric methods.

### CROSS-REFERENCE TO RELATED APPLICATIONS

The present application claims priority to: (1) U.S. Prov. Pat. App. No. 63/346,433, entitled Systems and Methods for Eye-Tracking Devices for Use in Hospital Settings, filed May 27, 2022; (2) U.S. Prov. Pat. App. No. 63/358,601, entitled System and Methods for Eye-Tracking Utilizing Distance Sensors, filed Jul. 6, 2022; (3) U.S. Prov. Pat. App. No. 63/358,603, entitled Systems and Methods for User-Specific Eye-Tracking Calibration Data and User Preferences, filed on Jul. 6, 2022; (4) U.S. Prov. Pat. App. No. 63/358,606, entitled Systems and Methods for Adjustable Infrared Illuminators, filed Jul. 6, 2022; (5) U.S. Prov. Pat. App. No. 63/388,376, entitled Networked Eye-Tracking Devices, filed Jul. 12, 2022; (6) U.S. Prov. Pat. App. No. 63/395,578, entitled Systems and Methods for Eye-Tracking Diagnostics, Calibration, and Gamification of Eye Exercises, filed on Aug. 5, 2022; (7) U.S. Prov. Pat. App. No. 63/395,580, entitled Systems and Methods for Improved Eye-Tracking User Interfaces, filed on Aug. 5, 2022; (8) U.S. Prov. Pat. App. No. 63/396,448, entitled Systems and Methods for Eye-Tracking with Non-Interactive Surfaces, filed on Aug. 9, 2022; and (9) U.S. Prov. Pat. App. No. 63/397,075, entitled Systems and Methods for Eye-Tracking Using Machine Learning Techniques, which was filed on Aug. 11, 2022, the entire contents of which are hereby incorporated by reference.

### TECHNICAL FIELD

The present invention relates, generally, to eye-tracking systems and methods and, more particularly, to the use of improved eye-tracking systems deployed in a variety of contexts using novel user interface techniques.

### BACKGROUND

Eye-tracking systems—such as those used in conjunction with desktop computers, laptops, tablets, virtual reality headsets, and other computing devices that include a display—generally include one or more illuminators configured to direct infrared light to the user's eyes and an image sensor that captures the images for further processing. By determining the relative locations of the user's pupils and the corneal reflections produced by the illuminators, the eye-tracking system can accurately predict the user's gaze point on the display.

While eye-tracking systems provide enormous benefits, prior art systems are unsatisfactory in a number of respects. For example, it is often the case that patients in hospitals and similar health care contexts are unable to effectively communicate vocally or by typing/writing messages. This may be due to pre-existing conditions, intubation, the presence of a feeding tube or oxygen mask, or severe bodily injury. While eye-tracking systems have been used in other contexts in which the user's ability to communicate is limited, no such system has been designed for the specific, unique use-cases encountered by patients in a hospital context. There is therefore a long-felt need for systems and methods that allow patients to communicate with hospital staff, family members, and other individuals in instances where their ability to communicate is limited. More generally, while traditional eye-tracking systems are deployed in the context of the display of a computing device, there are many instances in which tracking a user's gaze might be performed in connection with a simpler, non-interactive display.

In addition, eye-tracking typically depends on a knowledge of the position of the user relative to the display screen, which in turn requires the system to infer or otherwise compute the distance of the user's face from the image sensor used for gaze detection. In conventional eye-tracking system, such distance measurements are computed based on available two-dimensional data, and may vary in accuracy.

Furthermore, the use of eye-tracking systems generally requires the user to undergo a brief calibration step, in which the user may be asked to look at specific areas of the screen. This procedure allows the eye-tracking system to calibrate the various geometric computations that will later be performed during run-time. Similarly, the user (or others) may configure the system and/or specify user preferences that apply to one or more applications running on the eye-tracking device. While brief, the process of calibrating and specifying user preferences can be onerous to the user, particularly in a hospital or other healthcare setting.

In addition, while geometric, mathematical methods are traditionally used to model and determine gaze point location, there are a number of factors that can lead to ambiguity in such measurements, such as lighting conditions, ambiguities relating to eyeglasses, malformed eye regions, and the like. In such cases, it would be desirable to provide more robust methods for calculating the gaze point. For example, conventional eye-tracking systems typically use IR illuminators that are set to a single, default illumination value. As a result, the signal-to-noise ratio at the image sensor may vary depending upon ambient light conditions and other factors.

Finally, while the use of single eye-tracking devices in an environment is advantageous and offers many benefits, currently known eye-tracking systems are unsatisfactory in that they do not leverage the benefits of other networked devices (eye-tracking systems, mobile devices, etc.) present within the environment or available remotely via a network, such as the internet. Systems and methods are needed to overcome these and other limitations of the prior art.

### SUMMARY OF THE INVENTION

Various embodiments of the present invention relate to systems and methods for, inter alia, an eye-tracking device and associated software that together allow a patient to communicate with health-care providers (HCPs), family members, and other individuals via eye gaze technology. The system presents the patient with a hierarchical and intuitive set of graphical menus that, for example, allow the patient, via his or her eye-gaze location, to indicate basic needs (e.g., food, bathroom, bed adjustment), alert the nursing staff to an emergency situation, and/or indicate pain level associated with a specific part of the patient's body. In parallel, an administrator of the system may use traditional touch screen functionality to assist in calibration, select patient preferences, and otherwise configure the system for use.

In accordance with one embodiment, the system additionally provides language translation between the patient and the hospital staff members. That is, in cases where a patient uses eye gaze location to provide a message in their native language, the audio output from the device would be in English or other language comprehensible to hospital staff.

In accordance with another embodiment, improved systems and methods are provided for performing vision-related diagnostics during calibration (e.g., sensing strabismus and other such issues), and then using the eye-specific calibration for eye-tracking. In accordance with one embodiment, an incentive is provided to the user to improve eye alignment during eye tracking by, for example, adding blur when the system determines that the gaze points of the two eyes are not aligned, and reducing a blur effect when the eyes are aligned.

In accordance with another embodiment, improved systems and methods are provided for performing eye-tracking using one or more dedicated distance sensors—such as one or more time-of-flight sensors—to determine the distance of the user from the display. The improved accuracy of such sensors also increases the accuracy of the resultant gaze measurements, and also removes a portion of the computational load required by the eye-tracking system to compute such distances. The sensor may be a single-zone sensor, or may employ multiple zones to determine the distance of various anatomical features of the user (head, eyes, etc.).

In accordance with another embodiment, improved systems and methods are provided for performing storing, recalling, and activating user-specific calibration data and user preferences.

In yet another embodiment, improved systems and methods are provided for performing eye-tracking using a variety of machine learning techniques instead of, or in addition to, traditional geometric methods.

In other embodiments, improved systems and methods are provided for: improving IR illumination levels and/or lenses in eye-tracking systems; performing eye-tracking using a variety of novel user interface and text-to-speech techniques; and performing eye-tracking using a variety of networked devices available in the environment and/or remotely addressable via a network.

### BRIEF DESCRIPTION OF THE DRAWING FIGURES

The present invention will hereinafter be described in conjunction with the appended drawing figures, wherein like numerals denote like elements, and:

FIG. 1 is a conceptual block diagram illustrating an eye-tracking system in accordance with various embodiments;

FIGS. 2A and 2B illustrate the use of an eye-tracking system in accordance with various embodiments;

FIG. 3 illustrates a user's eyes and various characteristics thereof useful in describing the present invention;

FIG. 4A-C, 5A-C show a range of visual menus and options in accordance with various embodiments of the invention;

FIG. 6 is a conceptual overview of a non-interactive surface including graphics situated near and/or within the region defined by the eye-tracking module itself;

FIG. 7 is a conceptual overview of a non-interactive surface in accordance with one embodiment of the invention;

FIG. 8 is a flowchart illustrating a calibration method in accordance with one embodiment;

FIG. 9 shows, conceptually, the progressive improvement of the graphical representation of an object on the screen based on the user's improvement of eye alignment during eye tracking;

FIG. 10 illustrates an example multi-zone distance sensor in accordance with just one embodiment;

FIG. 11 illustrates a method in accordance with one embodiment of the present invention;

FIG. 12 is a flowchart depicting a method in accordance with one embodiment;

FIG. 13 illustrates an IR intensity level adjustment method in accordance with one embodiment;

FIG. 14 illustrates an alternate IR illuminator system in accordance with one embodiment;

FIG. 15 is a conceptual overview of an eye-tracking user interface in accordance with one embodiment;

FIG. 16 is a conceptual overview of an eye-tracking user interface in accordance with another embodiment;

FIG. 17 is a conceptual overview of an eye-tracking user interface in accordance with another embodiment;

FIG. 18 is a conceptual overview of an eye-tracking user interface in accordance with one embodiment; and

FIG. 19 is a conceptual overview of a network of eye-tracking devices and other components in accordance with one embodiment of the invention.

### DETAILED DESCRIPTION OF PREFERRED EXEMPLARY EMBODIMENTS

The present subject matter generally relates to improved eye-tracking systems and methods applicable in a variety of contexts and using a range of novel user interface techniques. In that regard, the following detailed description is merely exemplary in nature and is not intended to limit the invention or the application and uses of the invention described herein. Furthermore, there is no intention to be bound by any theory presented in the preceding background or the following detailed description. In the interest of brevity, conventional techniques and components related to eye-tracking algorithms, image sensors, machine learning systems, cloud computing resources, hospital communication systems, and digital image processing may not be described in detail herein.

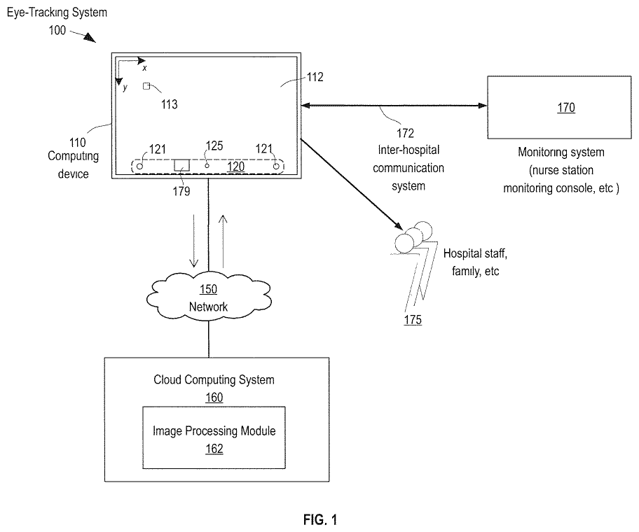

Referring first to FIG. 1 in conjunction with FIGS. 2A and 2B, an eye-tracking system 100 useful in describing the present invention includes a suitable form of computing device 110 (e.g., a desktop computer, tablet computer, laptop, smart-phone, head-mounted display, television panels, dashboard-mounted automotive systems, or the like) having an eye-tracking assembly 120 coupled to, integrated into, or otherwise associated therewith. Appropriate mounting hardware (200 in FIG. 2) may be provided to correctly position the eye-tracking system relative to the patient's face and eyes.

Eye-tracking system 100 is configured to communicate with one or more monitoring systems 170 (e.g., nurse stations, monitoring consoles, or the like) via any wired or wireless communication channel 172 (and associated protocol) now known or later developed. For example, channel 172 may consist of a proprietary inter-hospital communication system or a secure WiFi channel as is known in the art. While not illustrated in FIG. 1, eye-tracking system 100 may include suitable software/hardware (or be connected to such software/hardware) that provides an interface to the inter-hospital communication system.

As illustrated in FIG. 1, various hospital staff, family, and other appropriate individuals 175 are able to visually observe the state of eye-tracking system 100 as it is controlled by the patient, thereby providing a communication channel previously not available to patients with limited communication options. Similarly, the system 100 may also include an audio output that provides language translation between the patient and the hospital staff members. For example, the patient may use eye gaze location to provide a message in a first language, and the audio output from the device converts that message to a second language comprehensible to the hospital staff.

In general, the eye-tracking assembly 120 is configured to observe the facial region 181 (FIGS. 2A and 2B) of the patient (or “user”) 180 within a field of view 170 and, through techniques known in the art, track the location and movement of the user's gaze (or “gaze point”) 113, which may correspond to a location on a display (or “screen”) 112 of computing device 110, as illustrated. The gaze point 113 may be characterized, for example, by a tuple (x, y) specifying linear coordinates (in pixels, centimeters, or other suitable unit) relative to an arbitrary reference point on display screen 112 (e.g., the upper left corner, as shown). The distance of the user's facial region 181 from device 110 might also be measured. Similarly, the high speed movement and saccades of the user's pupil(s) may be sampled, in addition to the gaze itself.

In the illustrated embodiment, eye-tracking assembly 120 includes one or more infrared (IR) light emitting diodes (LEDs) 121 positioned to illuminate facial region 181 of user 180. Assembly 120 further includes one or more cameras 125 configured to acquire, at a suitable frame-rate, digital images (“eye-tracking image data,” “eye images,” or simply “images”) corresponding to region 181 of the user's face. This image data may be stored in any convenient lossy or lossless image file format, such as JPG, GIF, PNG, TIFF, RAW, or any other format known in the art. In addition—particularly in the context of cloud tracking—various video compression techniques may be used. Suitable video coding formats include, for example, H.264, H.265, VP9, VP10, and/or machine learning based image compression tailored to eye-finding applications. Furthermore, the image data may be further compressed and/or partitioned into packets—with associated metadata—for transmittal over a network (e.g., network 150).

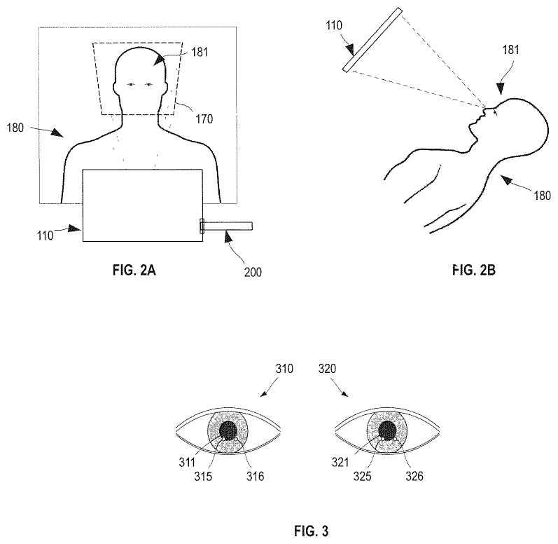

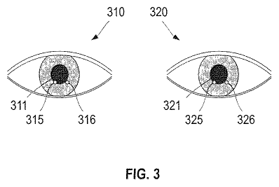

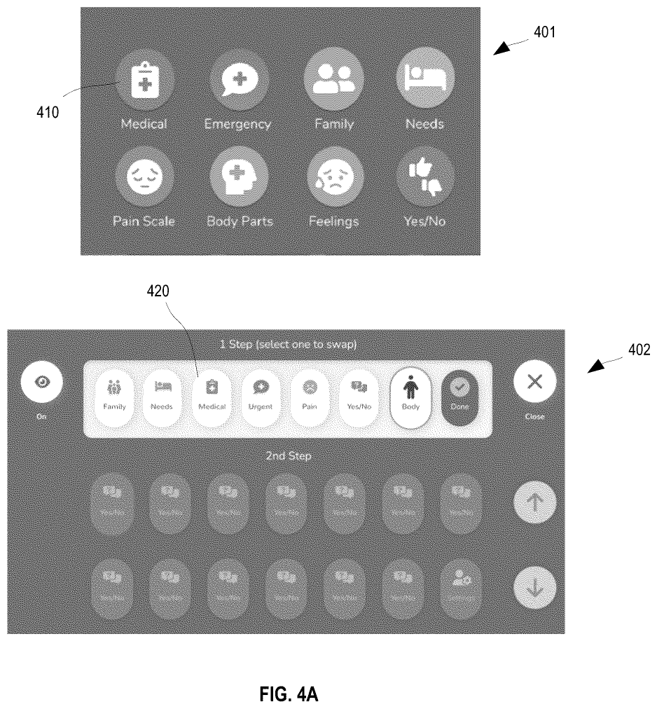

Referring to FIG. 3, gaze detection generally involves observing the pupil centers (PCs) and one or more corneal reflections (CRs) for each eye—e.g., PC 311 and CRs 315, 316 for the user's right eye 310, and PC 321 and CRs 325, 326 for the user's left eye 320, which are suitably determined using system 100. System 100 then processes the PC and CR data (the “image data”), as well as other available information (e.g., head position/orientation for user 180), and determines the location of the user's gaze point 113 on display 112. The gaze point 113 may be characterized, for example, by a tuple (x, y) specifying linear coordinates (in pixels, centimeters, or other suitable unit) relative to an arbitrary reference point on display screen 112. The determination of gaze point 113 may be accomplished in a variety of ways, e.g., through calibration methods or the use of eye-in-head rotations and head-in-world coordinates to geometrically derive a gaze vector and its intersection with display 112, as is known in the art.

In some embodiments, the image data is analyzed locally—i.e., by a processing system located within computing device (or simply “device”) 110 using a corresponding software client (referred to as “edge processing”). In some embodiments, however, processing of image data frames is accomplished using an image processing module (or “processing system”) 162 that is remote from computing device 110—e.g., hosted within a cloud computing system 160 communicatively coupled to computing device 110 over a network 150 (referred to as “cloud processing”).

During cloud processing, processing system 162 performs the computationally complex operations necessary to determine the gaze point from frames of image data and is then transmitted back (as eye and gaze data) over the network to computing device 110. An example cloud-based eye-tracking system that may employed in the context of the present invention may be found, for example, in U.S. patent application Ser. No. 16/434,830, entitled “Devices and Methods for Reducing Computational and Transmission Latencies in Cloud Based Eye Tracking Systems,” filed Jun. 7, 2019, the contents of which are hereby incorporated by reference.

Processing may take place in “real-time” mode or “buffered” mode. “Real-time processing,” as used herein, refers to a mode in which the system is processing eye images with a low enough latency between image capture and result that the user is provided with an interactive experience controlled by his or her eye gaze. For example, real-time processing is typically used with text communicators in which a non-verbal and/or mobility impaired individual gazes at cells containing words or letters to activate the cells and assemble messages that are then spoken aloud by the device's speakers. “Buffered processing,” in contrast, refers to a mode in which the system is capturing images at a relatively high rate (e.g., 120 Hz to 500+ Hz) with most of the system's resources prioritized for capture speed. Once capture is complete—or possibly while capturing is taking place—the eye tracking images are processed with the intent of returning one or more results that are based on processing batches of the images.

Having thus described an eye-tracking architecture in accordance with various embodiments, its manner of operation will now be described in conjunction with a set of exemplary visual menus as shown in FIGS. 3-5C. It will be appreciated that the menus illustrated in the figures are not intended to limit the invention in any way (e.g., in either function or form), and are only provided as examples.

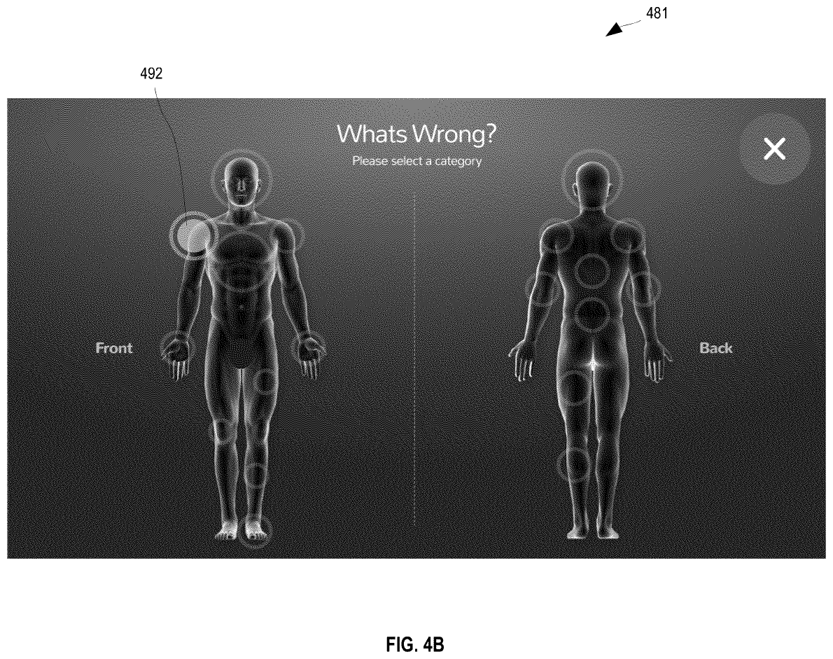

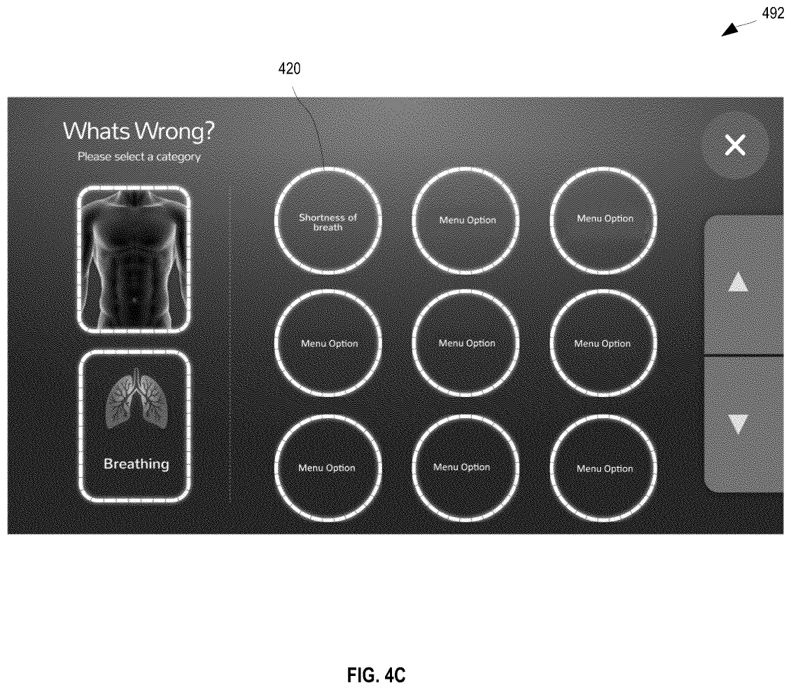

Referring first to FIG. 4A, an exemplary high-level menu 401 is shown. That is, the menu (e.g., 401) is intended as a “home” menu from which the patient can, via eye gaze, select from a set of high-level options (e.g., 410), including, in this embodiment: “Medical”, “Emergency”, “Family”, “Needs”, “Pain Scale”, “Feelings”, and “Yes/No”.

The “Emergency” option in this embodiment may be configured to quickly provide an emergency alert to hospital staff in the event that the patient is in distress and/or requires immediate attention. Thus, this option is similar to the traditional mechanically actuated buttons often provided to patients for just this purpose.

The “Family” option may be configured to allow communication with designated family members through, for example, a voice call, video conferencing, text messaging, email, or the like. Toward that end, the patient may be presented with a range of submenus including contact lists, communication methods, and a set of visual icons that the patient may choose from.

The “Needs” option, as mentioned briefly above, allows the patient to communicate (via one or more submenus) basic needs, such as food, bathroom access, room temperature, bed angle, and the like.

The “Pain Scale” option allows the user to visually indicate the level of pain that they are feeling. This may be done in a variety of ways using any convenient visual aids. In one embodiment, for example, the system includes one or more submenus that allow the patient, via eye-gaze, to indicate a position along a one-dimensional pain scale.

The “Body Parts” option allows the patient to highlight to hospital staff a concern over a particular anatomical region. Such an embodiment is shown in FIG. 4B, wherein the patient is first prompted (via graphical menu 481) to select “What's Wrong” by indicating a position relative to a graphical depiction of a body (in this case, the shoulder region 492).

As shown in FIG. 4C, once the patient selects an anatomical region, he or she may then select from a range of categories associated with that anatomic region (via graphical menu 492). In FIG. 4C, for example, the patient has first selected the chest region (upper right image), and “Breathing” (lower right image) and is then presented with a gallery of options to choose from, including “Shortness of breath” and other breathing-related problems. The “Body Parts” option may be used in conjunction with the “Pain Scale” option—i.e., allowing the patient to simultaneously highlight a body part while indicating a level of pain felt at that body part.

Referring again to FIG. 4A, main menu 401 may also include a “Feelings” option that allows the patient to communicate particular emotions (via either text or emojis), such as sadness, concern, happiness, reluctance, etc.

A “Yes/No” option (FIG. 4A, top) is provided to allow the patient to quickly answer yes-or-no questions posed by hospital staff (e.g., “are you comfortable,” “are you ready for breakfast,” etc.). Finally, a “Medical” option is provided in order to allow communication regarding other medical issues.

The menus in FIG. 5A-5C illustrate examples of administrative functions (operated, for example, via touch-screen) that allow hospital staff to configure the set of menu items that are presented to the patient. That is, particular menu items may be selected from a group of options that can be “swapped out” with others depending upon, for example, the nature of the injury, the nature of the patient, the nature of the treatment being provided, and the like.

In accordance with another embodiment, the present invention relates to the use of eye-tracking systems to determine gaze points on “non-interactive” surfaces. In this regard, the phrase “non-interactive surface,” in a non-limiting sense, means a surface (or region) with printed graphics/words/physical objects (“visual indicia”) that can be observed by a user, and which is not a computer monitor, tablet display, smartphone display, or other computer-controlled surface. In some embodiment, the visual indicia are printed on a surface. In other embodiments, the visual indicia is removable and/or projected onto a surface from a simple projector or the like. Stated another way, the eye-tracking system may be calibrated to determine the location of the user's gaze anywhere within the user's environment (i.e., not just a computer monitor or other interactive display).

In one embodiment, useful in a hospital and/or healthcare context, a passive surface is placed on the ceiling or otherwise above patient and includes a variety of options tailored to the patient's needs. Alternatively, the ceiling itself may be considered a blank, passive surface in which the available options are projected from a nearby projector module onto the ceiling for selection by the user. The projector may be “always on” (e.g., when the user is awake) or activated only when the patient presses a button, makes an audible command, or the like.

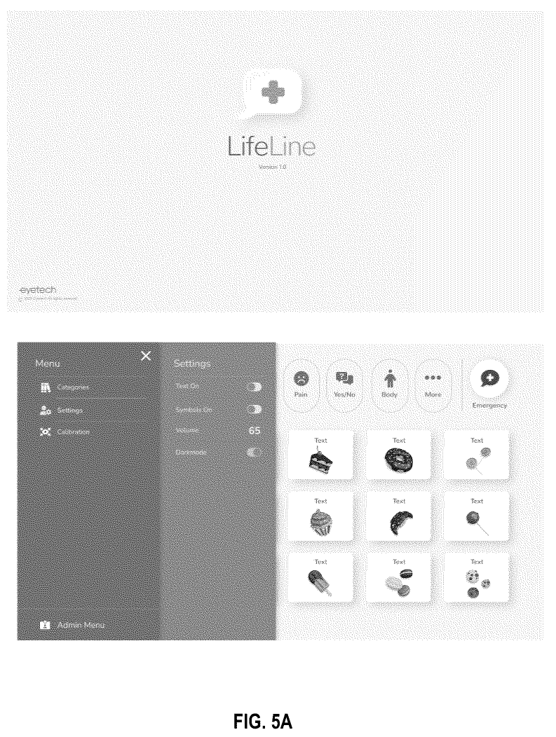

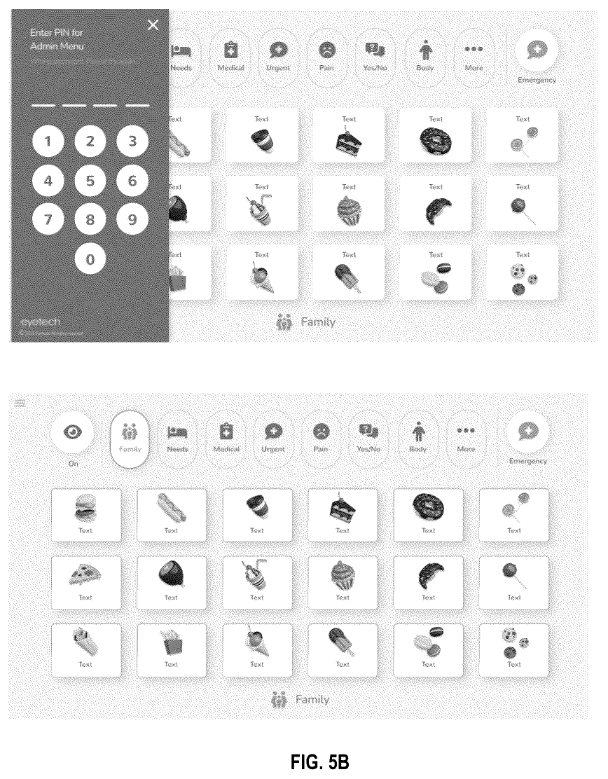

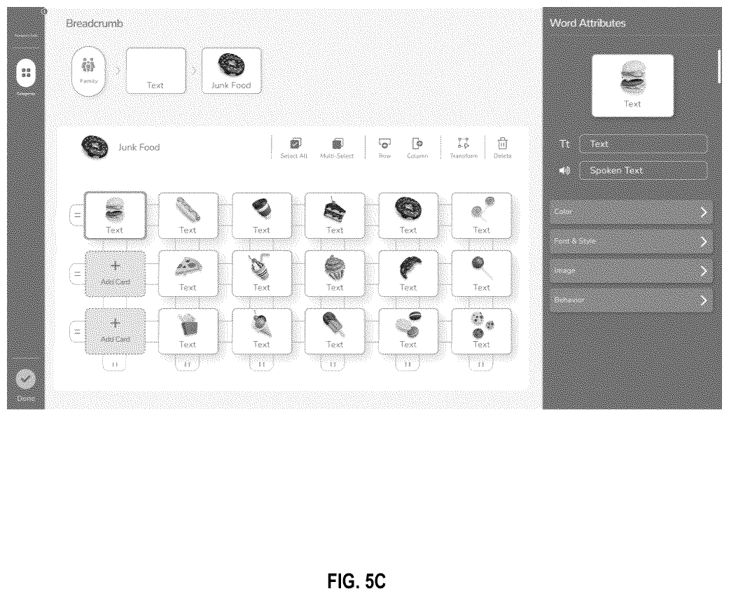

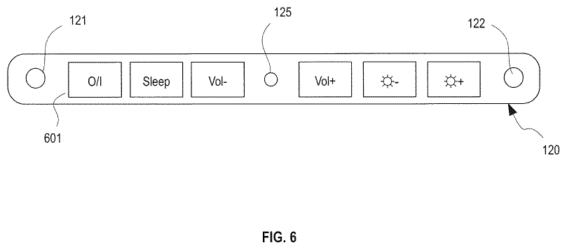

Referring to FIG. 6, in accordance with one embodiment the typically unused space between cameras (e.g., 125) and IR illuminators (e.g., 121, 122) on the eye-tracking module region 120 of a tablet or laptop is used as the non-interactive surface, and the eyes of the user are tracked within that region 120. Printed graphics/test 601 corresponding to commonly used functions (e.g., on/off, “sleep”, audio volume, screen brightness, etc.) are printed or otherwise provided within region 120 and can be interacted with by the user.

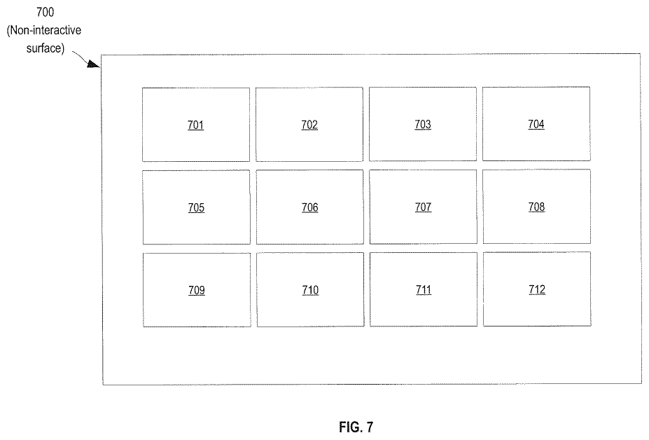

More generally, as shown in FIG. 7, a grid or matrix of options 701-712 may be provided on a non-interactive surface 700 and may be placed in any position or orientation observable by the user. It will be understood that the size, number, and shape of options shown in FIGS. 6 and 7 are not intended to be limiting, and that any number of visual indicia may be used, depending upon the application.

In some embodiments, the options provided in FIG. 7 include the ability to call for help within a facility. The system may further include gaze-implemented customizable functions (i.e., a visual area to define regions of functionality), as well as functionality to turn off room lighting/fans. In other embodiments, the system may take action outside of the normal function of the application by looking at an object rather than a button/control displayed within region 500. That is, the eye-tracking system may determine that the user's gaze corresponds to a thermostat in the room and subsequently allows the user to control its set-point using his or her eyes.

In another embodiment dealing with calibration procedures, the present invention has two related aspects—one directed to the acquisition of diagnostic vision data during the calibration process, and another directed to how the eye-tracking system can use that data.

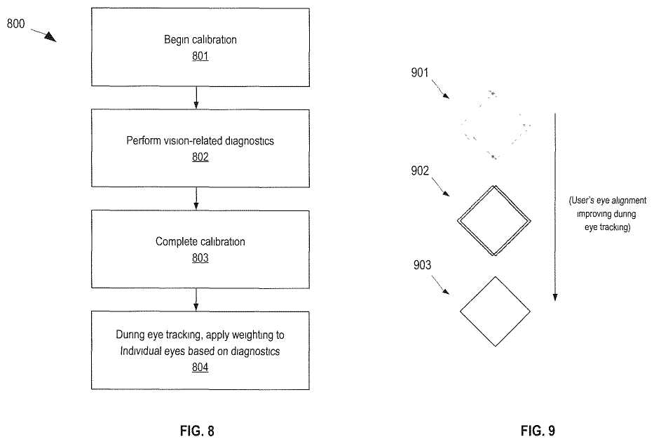

More particularly, referring now to FIG. 8, a method 800 in accordance with one embodiment generally includes beginning the eye-tracker calibration procedure (801) and during such calibration, performing vision-related diagnostics (802). In one embodiment, for example, it might be determined by the system that the user's eyes are misaligned—i.e., that they point to different gaze points that are spatially offset by some known distance or solid angle. This might be an indicator of strabismus, a malady in which one or more eyes deviate inward (esotropia) or outward (exotropia).

In such cases, the system may store individual calibration data for each eye, rather than averaging or otherwise accommodating this difference when performing the relevant calibration computation. That is, after calibration is complete (803), the diagnostic information may be used during normal eye-tracker operation (804) in order to improve the accuracy of eye tracking or to accomplish some other goal.

For example, the system may weigh one eye greater than another eye when it determines that the first eye's gaze point has a greater overall accuracy than that of the second eye. This weight may be applied in a variety of ways, depending upon the way eye-tracking calculations are performed. For example, the gaze point may be computed as a simple linear combination of the individual gaze points associated with each eye. This is not intended to be limiting however; any method of accommodating differences in eye behavior may be used.

In accordance with another embodiment, the gaze point calculation may be further refined by applying weights to individual CRs (two per eye) independently—i.e., using four separate eye-gaze data points.

In accordance with another embodiment of the invention, an incentive is provided to the user to bring his or her eyes together when it was previously determined (e.g., via step 402) that the user suffers from a vision problem. That is, the user effectively performs eye exercises during the eye-tracking process. This is particularly useful with respect to eye issues that are treatable via such eye exercises, such as strabismus, as discussed above.

The manner in which this visual incentive is provided may vary. In one embodiment, for example, a target image on the display screen may be purposely blurred based on the misalignment of the user's eyes (e.g., proportionate to the difference in gaze points) and then progressively “unblurred” as the user brings the eyes together. That is, the user is rewarded for performing an alignment exercise using some visual enhancement of the image or individual objects on the screen.

FIG. 9 illustrates just one example. Specifically, an object (represented by the diamond shapes (901-903)) is rendered out of focus and/or as a doubled object when the user's eyes are misaligned by some predetermined amount (901). The out-of-focus nature of the image incentives the user to bring his or eyes together, causing the corresponding graphical object to progress in focus from just slightly out of focus (902) to perfectly in focus (901). In some embodiments, the user is only allowed to “click” or otherwise choose an object when a certain level of eye alignment has been achieved.

The above visual incentive may take a variety of forms, and may be applied to any number of objects on the display screen (such as menu choices, boxes, graphics, etc.). In one embodiment, for example, only those objects that are within some specified distance of the gaze point(s) are treated in this way. In other embodiments, only objects within certain regions of the screen are affected.

In a particular embodiment, this procedure is used in the context of a computer game—literally “gamifying” the performance of eye exercises. For example, while playing a first-person-shooter game, the cross-hair used for aiming may be obscured, de-focused, or otherwise degraded based on the level of alignment of the user's eyes.

As described briefly above, the present invention provides improved systems and methods for performing eye-tracking using one or more dedicated distance sensors (e.g., distance sensor 170 in FIG. 1) to determine the distance of the user from the display.

Sensor 179 may employ any form of distance measurement technique known in the art. In one embodiment, for example, sensor 179 comprises a time-of-flight sensor, in which light pulses (or other such pulses) are transmitted toward the user, and the time necessary for these pulses to return to the sensor are used to determine distance. The present invention is not so limited, however, and may employ any form of distance sensors now known or later developed, such as lidar sensors, ultrasonic sensors, IR proximity sensors, acoustic sensors, and the like.

The sensor may be a single-zone sensor, or may be a multi-zone sensor capable of a more granular estimate of distance. That is, either a grid of distance sensors or a single sensor with multiple zones may be employed.

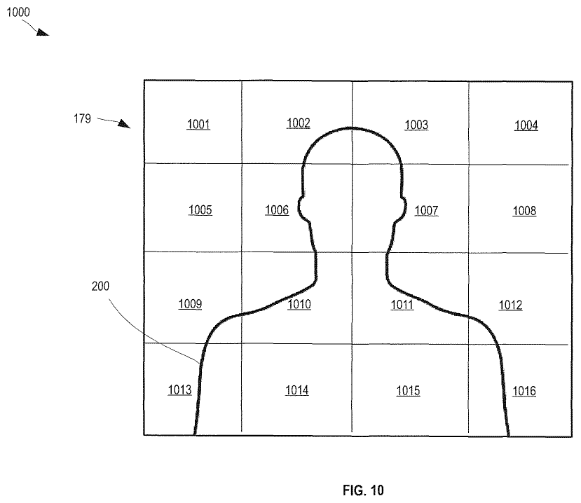

FIG. 10 illustrates, conceptually, an example multi-zone sensor 179 configured to determine the distance of a user (shown superimposed on the sensor regions for illustration purposes). In this embodiment, the sensor 179 has 16 zones configured in a 4×4 array (zones 1001-1016). Each zone is capable of producing its own estimate of distance. For example, zones 1002, 1003, 1006, 1007 will generally provide an estimate of the distance to the user's head and face, while zones 1010, 1011, 1014, and 1015 will generally provide an estimate of the distance to the user's upper body.

It will be appreciated that the embodiment shown in FIG. 10 is not intended to be limited, and the number of zones, and the field-of-view for the distance sensor itself, may be selected to achieve the desired result. For example, the field of view for the distance sensor may be narrowed to be more focused on the user's facial region (rather than encompassing portions of the user's body).

In some embodiment, the system may use the distance sensor 179 to determine the distance of multiple users from the display screen. That is, in cases in which two individuals are observing the screen at the same time, the system may distinguish between the two users and provide a distance measurement for each individual.

In accordance with various embodiments, the distance sensor 179 may be used to determine whether the user is present. In this way, the system provides power saving by going into a “sleep” mode or the like when no user is present within the field of view, and conversely waking up when a user enters the scene.

In accordance with various embodiment, the distance sensor may be used to recognize that multiple people are present within the environment, and may adjust the system's behavior accordingly. This is particularly useful in a hospital context, where it is common for a caregiver, family, and other individuals to be located in the same room as the user. For example, the volume of the device 110 may be increased when multiple people are determined to be within the environment.

In accordance with various embodiments, the distance sensor 179 is also able to recognize large scale gestures of the user's body, such as nodding in agreement, head-shaking in disagreement, hand motions, and the like.

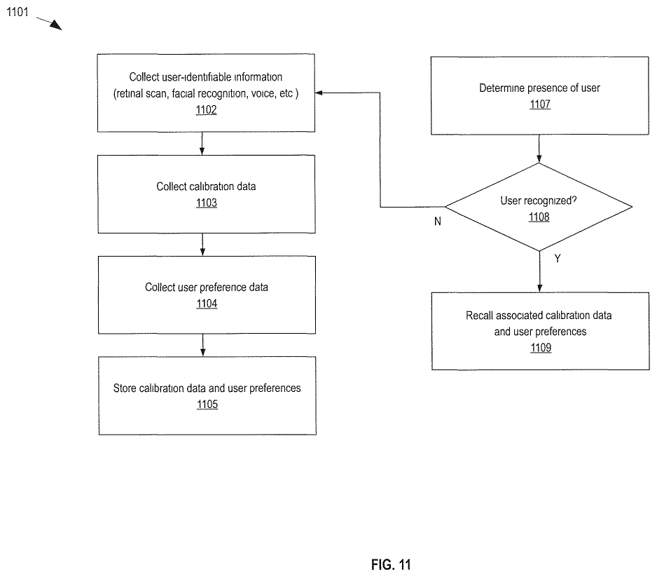

Various embodiments of the present invention also relate to improved methods for storing and recalling user-specific calibration and configuration data. More particularly, referring now to FIG. 11, the process 1100 begins by collecting any user-identifiable information that may be used to later uniquely associate the calibration settings and preferences with the user (step 1102).

In various embodiments, the user-identifiable information includes one or more types of biometric data acquired via the camera assembly and/or through other user-interface devices communicatively coupled to the eye-tracking device. Such biometric data may include, for example, iris scan data, retinal scan data, corneal (or other) eye feature data, facial images, finger print or finger vein data, voice data, and the like.

Next, any calibration data associated with this particular user is collected (step 1103), along with any user preference data (step 1104).

As used herein, “calibration” data includes any parameters, settings, variable assignments, or the like that are used by the eye-tracking system to determine gaze position. As will be appreciated by those skilled in the art, the calibration data may vary depending upon the method used by the eye-tracking system to make these computations. In general, however, the calibration data will include a variety of parameters related to measurements and geometry related to the user and the eye-tracking device itself (e.g., positions of the IR LEDs relative to the camera assembly, etc.)

As used herein, “user preference data” relates to any configuration or preference setting selected by or for the user that is used by any software that is running on the eye-tracking system (e.g., operating system, eye-tracking software, application software that interfaces with the eye-tracking software, etc.). Such user preference data might relate, for example, to language preferences, graphical preferences, screen brightness, menu options, etc.)

After calibration data and user preference data has been collected, it is stored (step 405) locally within the eye-tracking device and/or remotely in a suitable cloud-storage system. Preferably, such information is stored securely (e.g., encrypted in-transit and in-place). In accordance with various embodiments, the calibration and preference data is stored in such a way that it can be downloaded or transferred to another eye-tracking device with the same or similar characteristics. In this regard, the present invention provides a way to implement a “no-calibration” eye-tracking system, in that, once the calibration settings are known for a particular device, then it can be applied to other devices that have the same or similar geometrical characteristics.

With continued reference to FIG. 11, subsequently, when the user begins to use the eye-tracking system, it may first determine the presence of the user (step 1107). That is, the appropriate biometric or other readings will be performed on the user, and that data will be matched to any user-identifiable information that was previously stored in steps 1105.

The calibration and preference data can be recalled based on a number of factors (using either machine learning techniques or simple association rules), such as voice characteristics, context (hospital room vs. living room, etc.), audio questions/responses perceived in the environment (e.g., via natural language processing), and other such factors, as described above.

If the user is recognized (“Y” branch from step 1108), then the associated calibration data and user preferences are recalled, and the eye-tracking system continues using these settings (step 1109). Otherwise, if the appropriate settings cannot be found (“N” branch from Step 1108) then the system collects the appropriate information as specified at step 1102.

In accordance with another embodiment, the present invention relates to the use of machine learning and predictive analytics techniques to determine gaze data in place of, or to augment, traditional geometric computation.

In accordance with one embodiment, a hybrid approach is used for eye-tracking. That is, the system switches between traditional geometric computation (“TGC mode”) and ML mode depending upon a number of factors, such as lighting conditions, ambiguities related to eyeglasses, malformed eye region, etc. The system can then manage trade-offs between computational complexity and speed/accuracy.

In accordance with one embodiment, object detection and classification are performed using a “YOLO”-based (or similar) algorithm on time-series data such that a single neural network predicts bounding boxes and class probability directly from full images during one evaluation process. See, e.g., Redmon et al., “You Only Look Once: Unified, Read-Time Object Detection” (2016) https://arxiv.org/pdf/1506.02640v5.pdf (2016); “YOLO9000: Better, Faster, Stronger,” https://arxiv.org/pdf/1612.08242v1.pdf (2016); https://pjreddie.com/darknet/yolo/.

In addition to determining gaze location, the above YOLO methods may be used to collect time-series eye-tracking data, then analyze that data to find fixations, saccades, etc. for diagnostic purposes.

In accordance with one embodiment, the ML data may be further used to calibrate and/or improve the traditional geometric computation over time. That is, knowledge gained by the system during the ML-based eye-tracking may be used to refine the accuracy of the corresponding geometric computations.

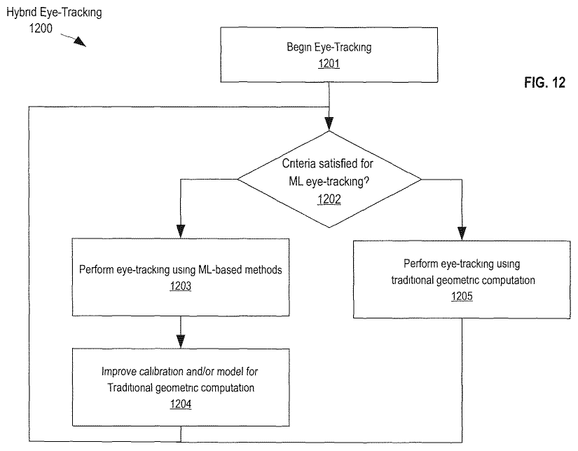

In that regard, FIG. 12 is a flowchart summarizing the above techniques and presents just one possible method 1200 for implementing a hybrid approach to eye tracking. Specifically, referring to FIG. 12, a method 1200 includes initiating (1201) the eye-tracking process (which might include a calibration step, as previously discussed), which would typically involve a traditional geometrical computation, but in some cases might default to machine learning techniques. During eye-tracking, the system then determines whether some predefined criteria have been satisfied such that the ML-based tracking may be preferred over traditional geometric computation (step 1202). If so, the system performs eye-tracking using the ML-based method (1203); otherwise, the system employs traditional geometric computation (1205).

Optionally, as shown in block 1204, the system may use the ML data to further calibrate and/or improve the model associated with traditional geometric computation. That is, any of the parameters used to model the geometry of the user's eyes, position, etc. may be modified to improve overall accuracy of the system. Subsequently, as shown, the system continues as before and continues to monitor the eye-tracking conditions and criteria. If it is determined that ML-based methods are no longer required, the system might choose to fall back to traditional geometric computation (e.g., to reduce computational complexity, etc.).

The decision step 1202 may take into account a wide range of environment-based and user-based conditions in selecting between ML and traditional geometric modes. Such conditions may include, for example, lighting conditions, ambiguities related to eyeglasses, malformed eye region, and/or other physical appearance variations of the user.

In addition to the use of YOLO for eye-tracking, the processing systems, modules, and other components described above may employ one or more additional machine learning or predictive analytics models to assist in carrying out their respective functions (i.e., with respect to bandwidth and latency criteria). In this regard, the phrase “machine learning” model is used without loss of generality to refer to any result of an analysis that is designed to make some form of prediction, such as predicting the state of a response variable, clustering, determining association rules, and performing anomaly detection. Thus, for example, the term “machine learning” refers to models that undergo supervised, unsupervised, semi-supervised, and/or reinforcement learning. Such models may perform classification (e.g., binary or multiclass classification), regression, clustering, dimensionality reduction, and/or such tasks. Examples of such models include, without limitation, stable diffusion, artificial neural networks (ANN) (such as a recurrent neural networks (RNN) and convolutional neural network (CNN)), decision tree models (such as classification and regression trees (CART)), ensemble learning models (such as boosting, bootstrapped aggregation, gradient boosting machines, and random forests), large language models, Bayesian network models (e.g., naive Bayes), principal component analysis (PCA), support vector machines (SVM), clustering models (such as K-nearest-neighbor, K-means, expectation maximization, hierarchical clustering, etc.), linear discriminant analysis models.

In accordance with one embodiment of the present invention, the intensity levels of IR LEDs 121 and 122 may be adjusted based on one or more sensed characteristics of the environment.

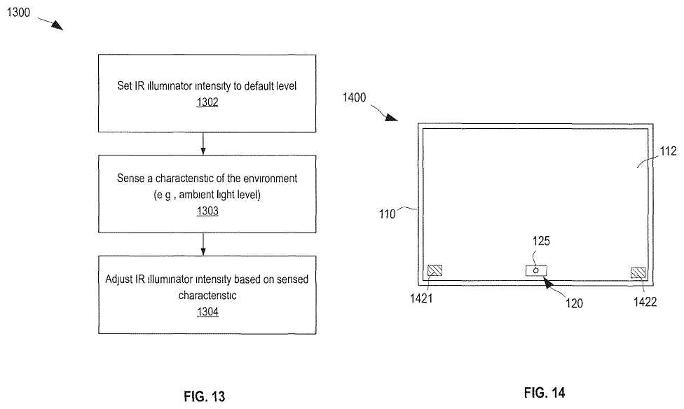

That is, referring to the conceptual flowchart 1300 shown in FIG. 13, the IR illuminators (e.g., 121 and 122 in FIG. 1) are set to some default level (step 1302)—which may be user-configurable or hard-wired into the software/hardware of the eye-tracking device. For example, the ambient light level might be sensed using a light sensor or camera assembly 125. Similarly, the intensity of the visible and/or IR light reflected off the user's face may also be determined.

Based on the sensed characteristics of the environment, the intensity of LEDS 121, 122 are adjusted (step 1304). For example, the IR illumination power may be increased when the system determines that the ambient light level is high, and decrease the IR illumination power when the system determines that the ambient light level is low. The magnitude of the illumination variation may be selected based on available hardware, but will generally be in the milliwatts per steradian level.

Referring to FIG. 14, another embodiment 1400 of the present invention generates IR light using one or more small lenses or film segments (1421, 1422) that are placed on the surface of the standard display screen 112. These segments 1421, 1422 are configured—by virtue of their structure and/or material properties—to convert a portion of the visible light passing therethrough to light in the IR spectrum. In this way, the cost and complexity of the eye-tracking module 120 can be reduced (i.e., by replacing costly IR LEDs with simple film or lens components).

In a further embodiment, rather than adding lenses or films to the surface of display 112, the system (via software/hardware) may be configured to modulate the intensity of small areas of the display screen (e.g., in regions 1421 and 1422) in a way that is detectible by camera 125. For example, the pixels in regions 1421 and 1422 may be changed quickly from one intensity level to another at a rate that is not perceivable by a human user, but can be sensed by camera 125 when viewing reflections in the user's eyes (thereby allowing the system to determine the corneal reflection points).

In accordance with another embodiment, camera assembly 125 includes a “steerable” lens that, among other things, allows the camera to adjust its field of view during operation. The steerable lens may also function to reduce vibration and provide other such benefits. In one embodiment, for example, the lens is electromagnetically controlled by computing device 110. Operation of the steerable lens may be coordinated in conjunction the intensity of the IR LEDs—i.e., to achieve some target signal-to-noise ratio.

In accordance with various embodiments of the present invention, novel user interface features are provided that are particularly applicable in an eye-tracking context. For example, a user is able to progress through a hierarchy of menu items that are displayed in a variety of ways depending upon the context.

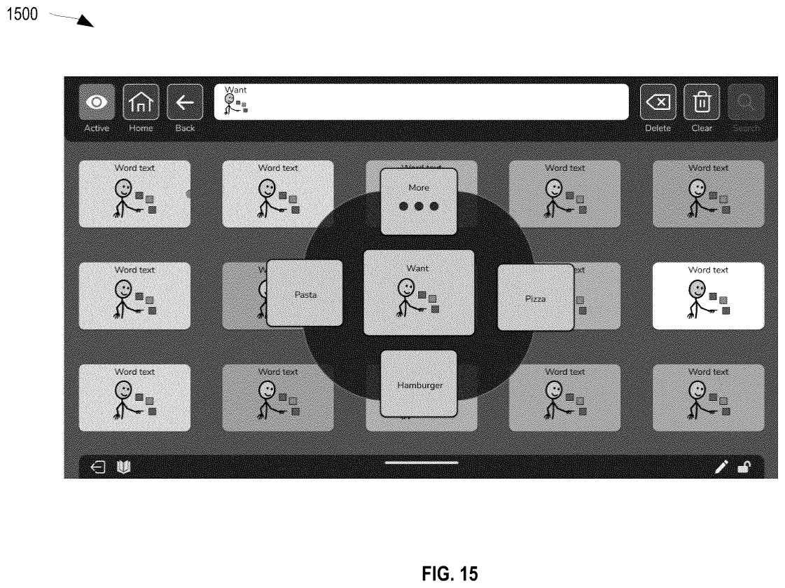

Referring now to FIG. 15, for example, a page of menu items 1500 may be displayed for the user in a matrix pattern (in this case, a 3×5 array of rectangular regions). When the user's gaze lingers on (or otherwise selects) one of the items, such as the “Want” item at the center of the display, two or more additional items are presented in the vicinity of the central item. This is an example of a “bloom” design—i.e., a context-specific design in which a series of secondary graphical elements are displayed circumferentially around the primary graphical element (like petals around the center of a flower), which themselves can be selected by the user. In the illustrated example, the central element (“Want”) is surrounded by the options “Pasta”, “Pizza”, “Hamburger”, and “More . . . ” (allowing the user to further describe what is wanted). A greyed-out region is also provided as a background, as shown, to help focus the user's attention. It will be appreciated that the content, number, shape, and position of the secondary graphical elements may vary depending upon the context, and that FIG. 15 is not intended to be limited.

In other examples, the primary text might be “Send Text” (or “Make Call”) and the secondary options might correspond to numbers within the user's contact list. Similarly, the central text might be “Pain”, and the secondary options might correspond to anatomical regions on the user's body. In another embodiment, the primary option is “Say”, and the secondary options are greetings or other messages which are then vocalized by the system (e.g., “Hi”, “Bye”, “No”, “Yes”, “Maybe”, etc.)

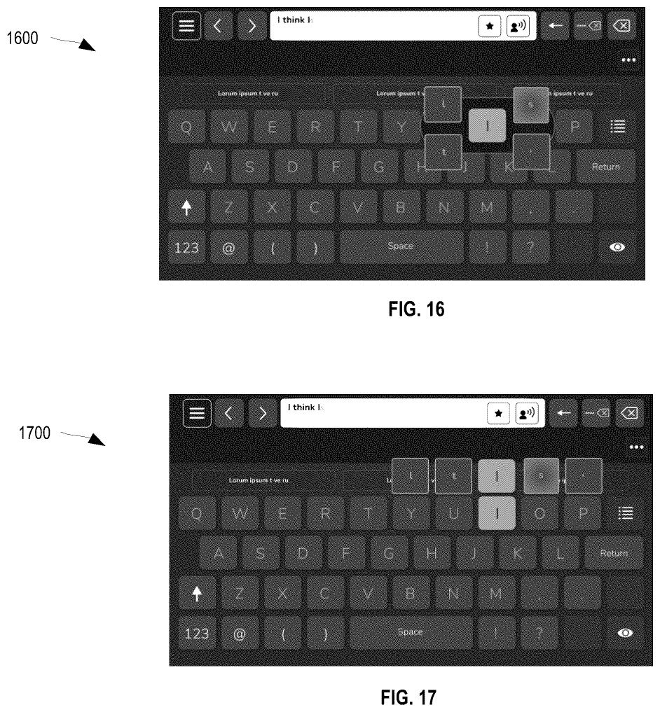

FIG. 16 illustrates another example 1600 that facilitates the selection of individual letters in a text string being “typed” by the user. As shown, the system provides a limited set of secondary characters (‘l’, ‘s’, ‘t’, etc.) around a primary character ‘l’. The secondary characters are chosen by the system based on maximum likelihood statistics or other machine learning techniques applicable to natural language processing. While only four secondary characters are illustrated in this embodiment, it will be appreciated that 5, 6, or even more such characters might be presented to the user, depending upon the context.

While FIG. 16 illustrates an example of a “bloom” design as described above (wherein the secondary characters are distributed circumferentially around a primary character), FIG. 17 illustrates another example 1700 in which the secondary characters are presented linearly above the primary character. It will be appreciated that this linear array may be oriented horizontally (as shown), vertically, or at any arbitrary angle. The number of secondary characters, as well as their sizes and positions, may vary depending upon the context.

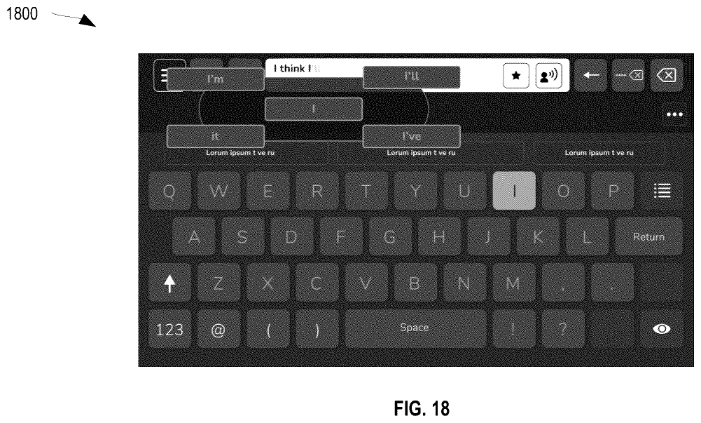

FIG. 18 illustrates yet another example 1800 in which a set of secondary words, rather than individual characters, are presented to the user. In this case, when the user selects the letter “I”, the system presents the options “I'm”, “I'll”, I've”, and “it”. This is another example of a “bloom” design, but in this case the central character is located near the text entry field at the top of the screen, rather than adjacent to the primary character (‘I’, in this example). In some embodiments, contextual word options are presented to the user based on the dynamic words entered previously as well as the category of speech (e.g., environment and location of the eye-tracking system).

While not shown in the examples, the above techniques (which are focused on the section of options or characters) may also be used to control the cursor function (e.g., right click/drag/left click, etc.) or indeed to perform any context-based action typically selected by a user with a mouse, keyboard, or other input device. For example, the range of options might correspond to the typical Windows right-click options.

In the context of text-to-voice conversion (which may apply to any of the preceding examples), the voice of the user may be sampled a priori so that text-to-voice conversion can be customized to the user's voice (e.g., AI voice training). This voice training step may be performed using actual text selections being spoken by the user, or (when the user is not currently able to speak) based on training data or available corpus of video/audio data taken from the user's Facebook, Instagram, YouTube channel, Tik Tok channel, or any other available recordings of the user's voice.

In some embodiments, the language used for text-to-voice translation (e.g., English, Spanish, German, etc.) may be selected automatically by the system based on context, geographical region, sensed language being used in the environment, and/or the like.

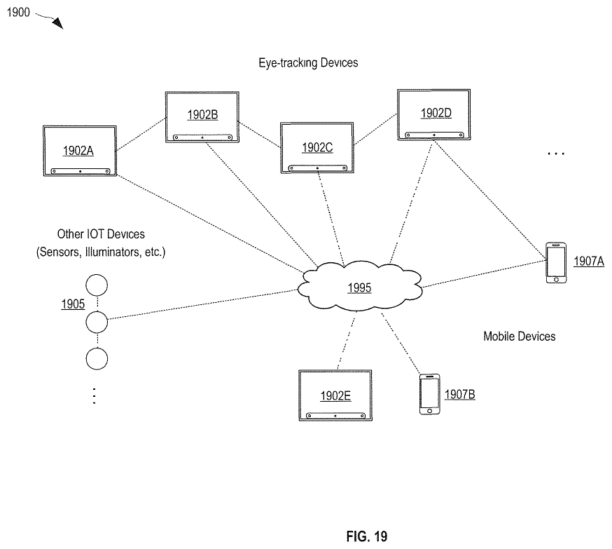

Referring now to FIG. 19, the present invention contemplates the use of multiple eye-tracking devices, mobile devices, and other networked components to provide an array of advantages. In general, as shown, an example eye-tracking network 1900 includes a variety of devices (e.g., IOT devices) interconnected via one or more data communication networks using any suitable network stack now known or later developed. As a preliminary matter, the example shown in FIG. 19 is not intended to be limiting with respect to the number, type, and topology of the various components that might be included in such a system.

In the illustrated embodiment, network 1900 includes any number of eye-tracking devices 1902 (e.g., 1902A-1902E), network infrastructure 1995, one or more mobile devices 1907 (e.g., 1907A and 1907B), and one or more additional internet-of-things (IOT) devices 1905. Eye-tracking devices 1902 may correspond, for example, to device 110 in FIG. 1, but may be any form of eye-tracking system. Mobile devices 1907 may comprise cell-phones, tablets, laptops, smart watches, or any other component capable of achieving the objects of the present invention. IOT devices 1905 may include, for example, individual IR LEDs, optical sensors, distance sensors, microphones, smart speakers, LED lights, smart TVs, and any other such IOT device).

In accordance with the present invention, the various components of network 1900 cooperate to improve the eye-tracking experience for both the user and other individuals associated with the user. For example, the systems and methods described herein are particularly applicable to a hospital or other healthcare context, as described more fully below.

In accordance with one embodiment, a mobile device 1907 is configured (through appropriate software and/or hardware) to communicate with one or more of the eye-tracking devices 1902 to monitor when and how the eye-tracking device 1902 is utilized by a user, which may include a “dashboard” that allows an individual to remotely determine in real-time the state of the user.

For example, in a health-care context, the user may correspond to a patient that is using an eye-tracking device during his or her stay in a hospital, and mobile device 1907 may be used by a doctor or other healthcare professional to monitor the behavior of the patient with respect to the eye-tracking device 1902. For example, to the extent that the eye-tracking device 1902 is capable of analyzing gaze data to determine possible cognitive impairment, such information may be shared with the healthcare professional as an alert or through other means. Similarly, the mobile device 1907 may be used by a family member of the patient to monitor any requests made by the patient via the eye-tracking device.

Multiple mobile devices 1907 may be configured to monitor a single eye-tracking device 1902, or conversely, multiple eye-tracking devices 1902 may monitored by a single mobile device 1907. Indeed, the entire network 400 may be monitored by one or more of the mobile devices 1907, with the information provided varying depending upon the role of the individual performing the monitoring (e.g., doctor role, nurse role, family member role, etc.)

In another embodiment, the IOT devices may be configured to produce gaze data for multiple individuals within an environment. That is, multiple IOT IR LEDs may be placed throughout the environment (on tables, walls, ceilings, etc.), along with corresponding IOT sensors, such that one or more processing units within network 1900 (e.g., any component with a processor) is capable of capturing gaze data.

In another embodiment, other networked devices may be connected via network infrastructure 1995, such as remote nurse stations, “teledoc” systems, and the like.

The processing systems, modules, and other components described above may employ one or more machine learning or predictive analytics models to assist in carrying out their respective functions. In this regard, the phrase “machine learning” model is used without loss of generality to refer to any result of an analysis that is designed to make some form of prediction, such as predicting the state of a response variable, clustering, determining association rules, and performing anomaly detection. Thus, for example, the term “machine learning” refers to models that undergo supervised, unsupervised, semi-supervised, and/or reinforcement learning. Such models may perform classification (e.g., binary or multiclass classification), regression, clustering, dimensionality reduction, and/or such tasks. Examples of such models include, without limitation, artificial neural networks (ANN) (such as a recurrent neural networks (RNN) and convolutional neural network (CNN)), decision tree models (such as classification and regression trees (CART)), ensemble learning models (such as boosting, bootstrapped aggregation, gradient boosting machines, and random forests), B ayesian network models (e.g., naive Bayes), principal component analysis (PCA), support vector machines (SVM), clustering models (such as K-nearest-neighbor, K-means, expectation maximization, hierarchical clustering, etc.), linear discriminant analysis models.

As used herein, the terms “module” or “controller” refer to any hardware, software, firmware, electronic control component, processing logic, and/or processor device, individually or in any combination, including without limitation: application specific integrated circuits (ASICs), field-programmable gate-arrays (FPGAs), dedicated neural network devices (e.g., Google Tensor Processing Units), virtual machines, electronic circuits, processors (shared, dedicated, or group) configured to execute one or more software or firmware programs, a combinational logic circuit, and/or other suitable components that provide the described functionality.

As used herein, the word “exemplary” means “serving as an example, instance, or illustration.” Any implementation described herein as “exemplary” is not necessarily to be construed as preferred or advantageous over other implementations, nor is it intended to be construed as a model that must be literally duplicated.

While the foregoing detailed description will provide those skilled in the art with a convenient road map for implementing various embodiments of the invention, it should be appreciated that the particular embodiments described above are only examples, and are not intended to limit the scope, applicability, or configuration of the invention in any way. To the contrary, various changes may be made in the function and arrangement of elements described without departing from the scope of the invention.

1. An eye-tracking system of the type used in connection with a patient in a healthcare context, the system comprising:
a computing device including: an illumination source configured to produce infrared light; a camera assembly configured to receive a portion of the infrared light reflected from the patient's face during activation of the infrared illumination source and produce a plurality of image data frames; a processing system communicatively coupled to the camera assembly and the illumination sources, the processing system configured to produce eye-movement data for the patient based on the plurality of image data frames;
a monitoring system communicatively coupled to the computing device through an inter-hospital communication system;
a user interface, deployed on the computing device, that is controllable by the patient through the eye-movement data, the user interface including a plurality of user interface elements, including at least an emergency user interface element configured to transmit a distress signal to the monitoring system.

2. The system of claim 1, wherein the user interface further includes a user interface element allowing the patient to communicate with designated family members through at least one of a voice call, video conferencing, text messaging, and email.

3. The system of claim 1, wherein the user interface further includes a user interface element that allows the patient to communicate a set of basic needs, including at least a food request and a bathroom request.

4. The system of claim 1, wherein the user interface further includes a user interface element allowing the patient to visually indicate a level of pain felt by the patient.

5. The system of claim 4, wherein the user interface includes a submenu that allows the patient to indicate a position along a one-dimensional pain scale.

6. The system of claim 1, wherein the user interface further includes a user interface element allowing the patient to graphically highlight a particular anatomical region on the patient's body.

7. The system of claim 6, wherein the user interface further allows to select a range of categories associated with the highlighted anatomical region.

8. The system of claim 7, wherein at least one of the categories includes a pain scale indicator manipulatable by the patient.

9. The system of claim 1, wherein the user interface includes user interface elements corresponding to a ‘yes/no’ answer.

10. The system of claim 1, wherein the computing device further includes an administrative function that allows hospital staff to configure the user interface based on a set of predetermined criteria.

11. A hybrid machine learning eye-tracking system comprising:
a computing device including: an illumination source configured to produce infrared light; a camera assembly configured to receive a portion of the infrared light reflected from the patient's face during activation of the infrared illumination source and produce a plurality of image data frames; a processing system communicatively coupled to the camera assembly and the illumination sources, the processing system configured to produce eye-movement data for the patient based on the plurality of image data frames;
a hybrid eye-tracking module configured to perform eye-tracking via both geometric computation and machine learning, the hybrid eye-tracking module configured to: determine whether predetermined criteria are satisfied; perform geometric computation if the predetermined criteria are not satisfied; perform machine learning eye-tracking and improve a model for the geometric computation if the predetermined criteria are satisfied.

12. The system of claim 11, wherein improving the model includes modifying at least one parameter associated with the geometry of a user's eyes.

13. The system of claim 11, wherein the predetermined criteria includes lighting conditions.

14. The system of claim 13, wherein the predetermined criteria includes presence of eye-glasses.

15. The system of claim 14, wherein the predetermined criteria includes physical appearance of a user.

16. The system of claim 11, wherein, when the system performs machine learning eye-tracking, object detection and classification are accomplished via an algorithm in which a signal neural network predicts bounding boxes and class probability directly from full images during a single evaluation process.

17. The system of claim 16, wherein the system further collects time series eye-tracking data and analyzes the time-series eye-tracking data to determine at least one of fixations and saccades for the user's eyes.

18. The system of claim 17, wherein the system further uses the analyzed time-series eye-tracking data for diagnostic purposes.
